(19) United States
(12) Patent Application Publication
(10) Pub. No.: US 2006/0283652 A1
(43) Pub. Date: Dec. 21, 2006

(54) Biosignal detection device

(75) Inventors: Kenichi Yanai, Nisshin-city (JP); Tatsuya Ikegami, Nisshin-city (JP); Shinji Nanba, Kariya-city (JP)

Correspondence Address: POSZ LAW GROUP, PLC, 12040 SOUTH LAKES DRIVE, SUITE 101, RESTON, VA, 20191, US

(73) Assignee: DENSO CORPORATION, Kariya-city (JP)

(21) Appl. No.: 11/453,228

(22) Filed: Jun. 15, 2006

(30) Foreign Application Priority Data
Jun. 15, 2005 (JP) 2005-175013

(51) Int. Cl. B60K 28/00; G08B 23/00; A61B 5/103

(52) U.S. Cl. 180/272; 340/576; 600/587

(57) ABSTRACT

Pressure sensors are sorted out from the other sensors based on a signal from each sensor element. Sensor outputs of the pressure sensors that have been sorted out are filtered using an FIR filter through which sensor outputs of the other sensors are eliminated. Frequency analysis is performed on the filtered sensor outputs using FFT. A reference sensor is chosen from power spectra of the filtered pressure sensors. Phase differences are calculated between a sensor output of the reference sensor and the sensor outputs of the other pressure sensors. Based on the phase differences, pressure sensors other than the reference sensor are sorted into those with large phase differences and those with small phase differences. Phases of sensor signals of those with large phase differences are reversed, and their sensor outputs are added together. For those with small phase differences, their sensor outputs are added together without reversing the phases.

### CROSS REFERENCE TO RELATED APPLICATION

This application is based on and incorporates herein by reference Japanese Patent Application No. 2005-175013 filed on Jun. 15, 2005.

### BACKGROUND OF THE INVENTION

1. Field of the Invention

The present invention relates to a biosignal detection device, which detects biological information of a vehicle occupant, such as a driver, passengers or the like.

2. Description of Related Art

Various techniques have been disclosed as methods for detecting biological information, such as, a heart rate or a respiratory rate of those who are driving or sleeping.

For example, according to JP-2001-145605-A (corresponding to EP-1247488-A1), an internal pressure of an air bag disposed under bedclothes can be detected using a microphone, a pressure sensor or the like. The biological information, such as the heart rate, the breath, body motion or the like, is obtained by means of frequency analysis of the internal pressure signal.

According to JP-6-197888-A (corresponding to U.S. Pat. No. 5,574,641), an infrared heart rate sensor is placed on a driver's arm or the like in order to prevent a snooze. The heart rate of the driver is sensed using the heart rate sensor signal.

Furthermore, according to JP-3098843-B2, a device for detecting the heart rate is placed in a driver's seat, and a device for detecting sensitivity is placed elsewhere. A heart rate signal is detected by means of signal process after a sensitivity detection signal is subtracted from a heart rate detection signal based on outputs from the above devices.

However, there is a problem in that although the art disclosed in JP-2001-145605-A above is effective in detecting the biological information in a room with a little disturbance noise, yet in a vehicle interior while driving, for example, the heart rate signal or the like cannot be detected in a case of signals, which show overlapping of frequencies.

As regards the art disclosed in JP-6-197888-A above, there is a problem of poor availability and usability since the driver needs to wear the infrared heart rate sensor.

Furthermore, in the art disclosed in JP-3098843-B2 above, when a sensor itself contains noises, vibration transfer functions differ because a noise detection position is different from a heart rate detection position. Therefore, the noise elements cannot be eliminated even if the noises are subtracted from a heart rate detection sensor. In addition, it is not clear whether signals in the element that is difference calculated by the subtraction are attributed to the heart rate signal, or to the noises that have been left due to the inadequate noise subtraction.

### SUMMARY OF THE INVENTION

The present invention addresses the above disadvantages. Thus, it is an objective of the present invention to provide a biosignal detection device, which can detect biological information effectively without restraining a vehicle occupant.

To achieve the objective of the present invention, there is provided a biosignal detection device including a plurality of pressure sensors and a controller. The plurality of pressure sensors are arranged at a seat of a vehicle to sense a pressure of a body of a vehicle occupant when the vehicle occupant is present on the seat. The controller detects biosignal that is relevant to a human body activity of the vehicle occupant present on the seat. The biosignal is detected based on a measurement of at least one effective pressure sensor that is selected from the plurality of pressure sensors. The at least one effective pressure sensor is selected from the plurality of pressure sensors in such a manner that the measurement of the at least one effective pressure sensor is less than a first predetermined pressure and is greater than a second predetermined pressure. The second predetermined pressure is less than the first predetermined pressure.

To achieve the objective of the present invention, there is also provided a biosignal detection device including a plurality of pressure sensors and a controller. The plurality of pressure sensors are arranged at a seat of a vehicle to sense a pressure of a body of a vehicle occupant when the vehicle occupant is present on the seat. The controller detects biosignal that is relevant to a human body activity of the vehicle occupant present on the seat. The biosignal is detected based on measurements of multiple effective pressure sensors that are selected from the plurality of pressure sensors. The controller estimates a position of a heart of the vehicle occupant based on the measurements of the multiple effective pressure sensors when the vehicle occupant is present on the seat.

### BRIEF DESCRIPTION OF THE DRAWINGS

The invention, together with additional objectives, features and advantages thereof, will be best understood from the following description, the appended claims and the accompanying drawings in which:

FIG. 1 is a schematic view that depicts arrangement of detection air bags employed in a biosignal detection device according to a first embodiment of the present invention;

FIG. 2 is a schematic view that depicts a system configuration of the biosignal detection device according to the first embodiment;

FIG. 3 is a block diagram that indicates an electrical configuration of the biosignal detection device according to the first embodiment;

FIG. 4 is a flowchart that indicates a process performed in the biosignal detection device according to the first embodiment;

FIG. 5 is a diagram that indicates a power spectrum of a sensor output according to the first embodiment;

FIG. 6 is an illustrative diagram that indicates a method for eliciting a phase difference between sensors according to the first embodiment;

FIG. 7 is a schematic view that depicts an application of a device for analyzing asleep condition according to the first embodiment;

FIG. 8 is a flowchart that indicates a routine of calculating pulse wave propagation velocity, which is employed in the biosignal detection device according to a second embodiment;

FIGS. 9A and 9B are illustrative diagrams that indicate a method for eliciting peak arrival time according to the second embodiment;

FIG. 10 is a flowchart that indicates a routine of calculating a driver's heart position, which is employed in the biosignal detection device according to the second embodiment;

FIG. 11 is a flowchart that indicates a main routine employed in the biosignal detection device according to the second embodiment; and

FIG. 12 is a schematic view that depicts a system configuration of the biosignal detection device according to a third embodiment.

### DETAILED DESCRIPTION OF THE INVENTION

Embodiments of the present invention will be described below with reference to the accompanying drawings.

### First Embodiment

A biosignal detection device according to a first embodiment detects biological information, for example, a heart rate of a driver sitting on a driver's seat or the like, and assesses the driver's drowsiness and/or stress on the basis of the biological information.

First, system configuration of the biosignal detection device of the present embodiment will be described below.

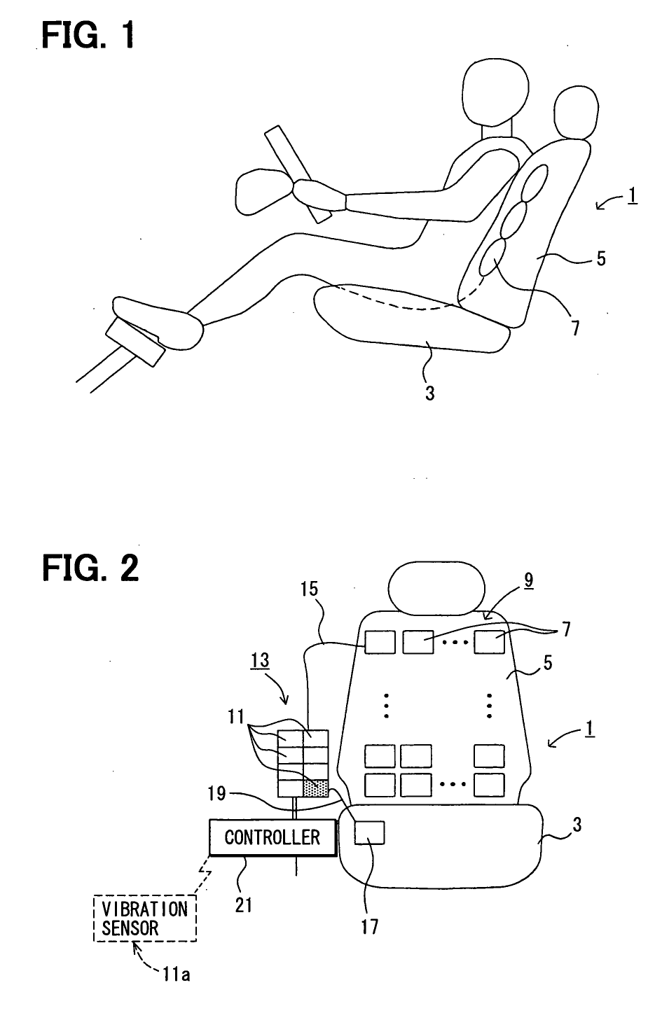

As shown in FIG. 1, a driver's seat 1 includes a seating face portion 3 on which the driver sits and a backrest portion 5 that supports the driver's back. The biosignal detection device of the present embodiment is disposed mainly in the driver's seat 1.

A plurality of bags that include air (i.e., detection air bags 7) is placed in the backrest portion 5 of the driver's seat 1. More specifically, the detection air bags 7 are arrayed in a grid pattern both along and all over a surface of the backrest portion 5 to form a back array 9 including air as shown in FIG. 2. This arrangement allows the detection air bags 7 to cover a wide range of pressure distribution, which leads not only to an understanding of all sensor outputs that indicate biological body activities of a human body, but also to an understanding of, for example, posture of the human body and the like. Additionally, each detection air bag 7 forms a detection part of the pressure sensor.

A sensor array 13 aside of the driver's seat 1 includes sensor elements 11 (of the pressure sensor) that are arrayed in a grid pattern. Pressure applied to the detection air bags 7 is transformed into electric information by the sensor elements 11, each of which may include, for example, a condenser microphone, a differential pressure sensor, or the like.

Each detection air bag 7 is connected to a corresponding one of the sensor elements 11 via a corresponding air tube 15 in such a manner that the detection air bag 7 has a one-to-one relationship with the sensor element 11. Consequently, each sensor element 11 can detect the pressure in each corresponding detection air bag 7.

Each pressure sensor includes the corresponding detection air bag 7 (or reference air bags 17), the corresponding sensor element 11, and the corresponding air tube 15. When the driver's back imposes a load on the backrest portion 5, the detection air bags 7 in the backrest portion 5 are compressed by its pressure. The pressure in each detection air bag 7 is transmitted to the corresponding sensor element 11 through the corresponding air tube 15 to detect the pressure (therefore, the load).

There are regions of the seating face portion 3 in which the pressure does not change when the driver sits on the portion 3. In such regions, similar bags the reference air bags 17 including air are arrayed to be employed as vibration detection sensors (a vibration reference). Likewise, each reference air bag 17 is connected to a corresponding one of the sensor elements 11 via a corresponding air tube 19.

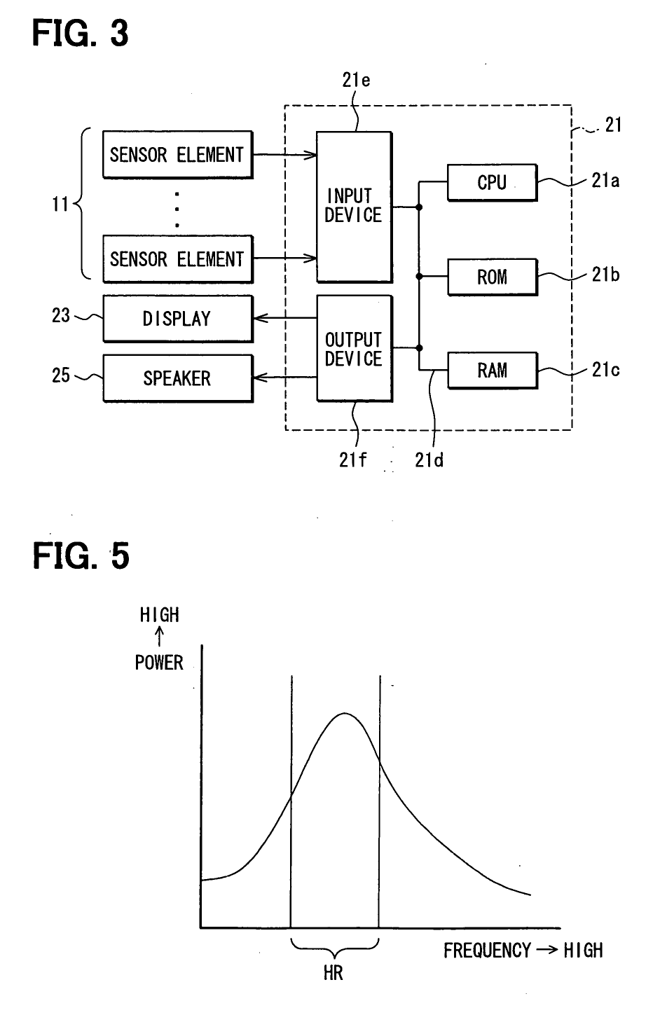

The pressure sensors (therefore, the sensor elements 11) are connected to an electronic controller 21. As shown in FIG. 3, the electronic controller 21 includes a widely known microcomputer as a main component thereof. The electronic controller 21 includes, for example, a CPU 21a, a ROM 21b, a RAM 21c, a bus 21d, an input device 21e, and an output device 21f. The input device 21e is connected to each sensor element 11, and the output device 21f is connected to a display 23 and a speaker 25.

Next, a process that takes place in the electronic controller 21 will be described below.

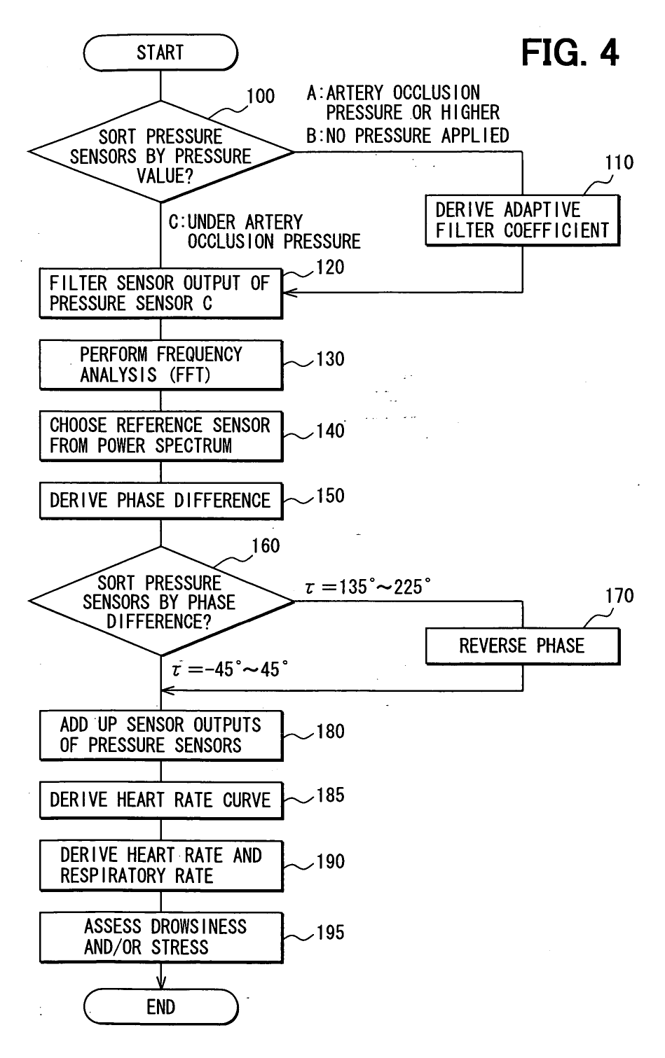

With reference to a flowchart of FIG. 4, at step 100, the pressure sensors are sorted based on a signal sent from each sensor element 11 to the electronic controller 21.

More specifically, when a pressure value of any pressure sensor is that of artery occlusion pressure or higher (hereafter a pressure sensor A) or that of no pressure applied (hereafter a pressure sensor B), control proceeds to step 110. When a pressure value of the pressure sensor is more than 0 (zero) yet under the artery occlusion pressure (hereafter a pressure sensor C), control proceeds to step 120.

This is due to the fact that in the case of the pressure sensor A and the pressure sensor B, a sensor signal that corresponds to the pressure applied by a biological body activity, for example, by the driver's heart rate, cannot be obtained. As regards the pressure sensor C, the sensor signal that corresponds to the pressure applied by the driver's biological body activity (i.e., pressure fluctuation due to the heart rate) can be obtained.

At step 110, an FIR filter, which eliminates sensor outputs of the pressure sensor A and of the pressure sensor B, is constructed, i.e., is designed. More specifically, an adaptive filter coefficient (a parameter) of the FIR filter is chosen such that the sensor outputs of the pressure sensor A and of the pressure sensor B are eliminated. As a method for eliciting the adaptive filter coefficient, a widely known LMS algorithm, for example, can be employed.

At step 120, the sensor output of the pressure sensor C is filtered through the FIR filter designed at the previous step 110. Like signals due to vibration of a vehicle or the like, the sensor output of the pressure sensor A and the pressure sensor B may mostly be other signals than those due to pressure applied by the driver's biological body activity. Therefore, the sensor output of the pressure sensor C is filtered through the FIR filter that eliminates the sensor outputs of the pressure sensor A and of the pressure sensor B. As a result, the pressure applied by the driver's biological body activity can exclusively be extracted from the sensor output of the pressure sensor C.

At step 130, frequency analysis is performed on the filtered sensor output by means of FFT (a fast Fourier transform). As a consequence of the frequency analysis, a power spectrum as indicated in FIG. 5, for example, is provided for each pressure sensor C in a case where the multiple pressure sensors C are present. In FIG. 5, HR (a heart rate) represents a range of frequencies indicating a state of the driver's heart rate (i.e., a heart rate frequency) and ranges from 0.7 Hz to 1.8 Hz.

At step 140, a reference pressure sensor C (a reference sensor) is chosen from the power spectra of the multiple pressure sensors C. For example, the pressure sensor C, which has the highest peak (or the largest integration value) of its power spectrum within the range of HR, may be defined as the reference sensor.

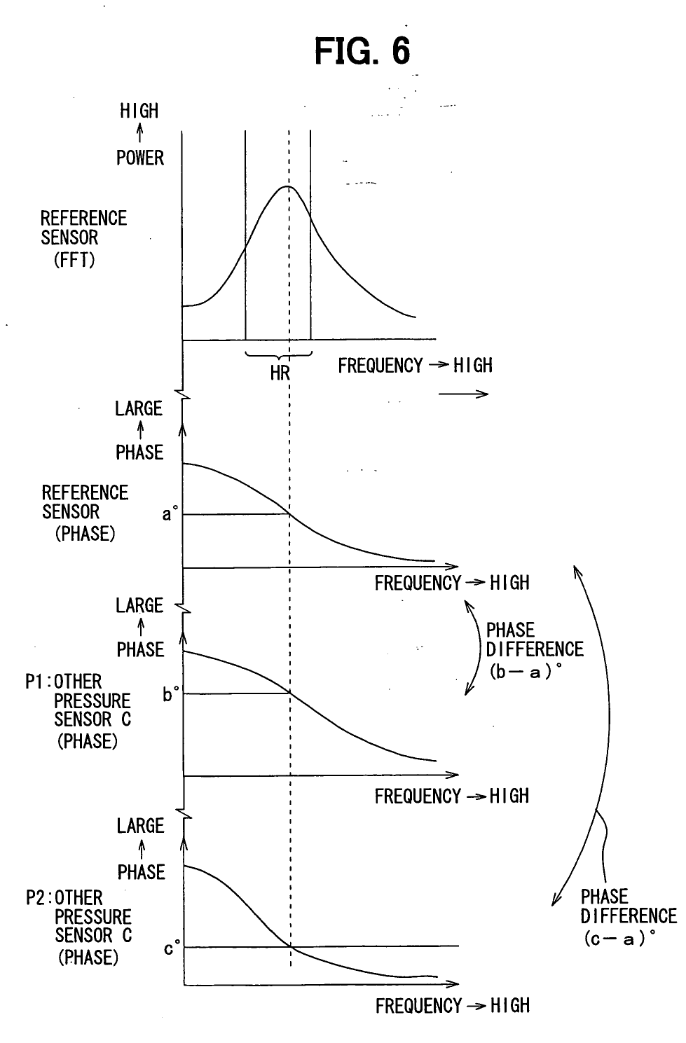

Since the pressure sensors are distributed over the surface of the driver=3 s seat 1, the signals that stem from the heart rate, for example, have different phases between their sensor outputs according to their distances from a driver's heart position. At step 150, phase differences (τ) between the sensor output of the reference sensor and that of the other pressure sensors C are evaluated. More specifically, on the basis of results of the FFT frequency analysis at step 130, the phase differences are evaluated as illustrated in FIG. 6. That is, phases of the other pressure sensors C (for example, P1, P2 in FIG. 6) that correspond to a phase at which the reference sensor has the maximum power spectrum (i.e., a° in FIG. 6) are calculated (i.e., b°, c° in FIG. 6 respectively). Furthermore, the differences from the phase a° are calculated (i.e., (b−a)°, (c−a)° in FIG. 6 respectively).

At step 160, other pressure sensors C, which are other than the reference sensor, are sorted according to the phase differences (τ). More specifically, when the phase differences (τ) are relatively large (e.g., the differences ranging from 135° to 225°), control proceeds to step 170. When the phase differences (τ) are relatively small (e.g., the differences ranging from −45° to 45°), control proceeds to step 180 as indicated in FIG. 4.

The phase of the sensor signal of the other respective pressure sensor C having the relatively large phase difference (τ) is reversed at step 170. By performing this correction to reduce the phase differences, elements of the signals that stem from the heart rate increase.

At step 180, with respect to the sensor output of the reference sensor, the sensor outputs of the other pressure sensors C are added up.

More specifically, when the phase differences (τ) are relatively small (e.g., −45° to 45°), the sensor outputs of the other pressure sensors C are added together without reversing the phases. When the phase differences (τ) are relatively large (e.g., 135° to 225°), the sensor outputs of the other pressure sensors C are added together after reversing the phases at step 170. The above addition is performed since a plurality of sensor outputs can be employed to improve measurement accuracy.

At step 185, a heart rate curve (a heart rate waveform) that indicates a change in the driver's heart rate is calculated using the sensor outputs that have been added up. At step 190, RRI (a heartbeat interval) and the heart rate are derived from the heart rate waveform.

At step 195, the driver's drowsiness and/or stress are assessed using the RRI and the heart rate. Since methods for assessing the drowsiness and/or the stress according to the RRI and the heart rate are widely known, the description thereof is omitted. For reference, the methods according to, for example, JP-6-197888-A and JP-2003-290164-A can be employed for the drowsiness and/or stress assessment.

As described above, in the present embodiment, the detection air bags 7 are arrayed in the grid pattern in the backrest portion 5 of the driver's seat 1 in order to detect the pressure in the detection air bags 7 as a result of the load imposed on the backrest portion 5 by the driver's back (see FIGS. 1 and 2). Furthermore, by sorting the sensor outputs of the pressure sensors, the pressure sensors that can detect the driver's heart rate are selected while elements of signals other than the heart rate signals are eliminated. Therefore, despite the driver's unrestrained conditions, the heart rate and the heartbeat interval can be measured accurately. The driver's drowsiness, stress or the like can be assessed appropriately on the basis of results of the accurate measurement.

Additionally, methods (1)-(3) below can be employed as applications of the first embodiment.

(1) A vibration sensor (e.g., a G sensor) (not shown) can be substituted for the reference air bag 17 and its corresponding pressure sensor.

In this case, in order to match a sensor signal of the vibration sensor with that of the pressure sensor, a predetermined transfer function can be used for processing the sensor signal of the vibration sensor. Similar to the first embodiment, the FIR filter is developed by eliciting the adaptive filter coefficient, so that sensor output of the vibration sensor can be eliminated.

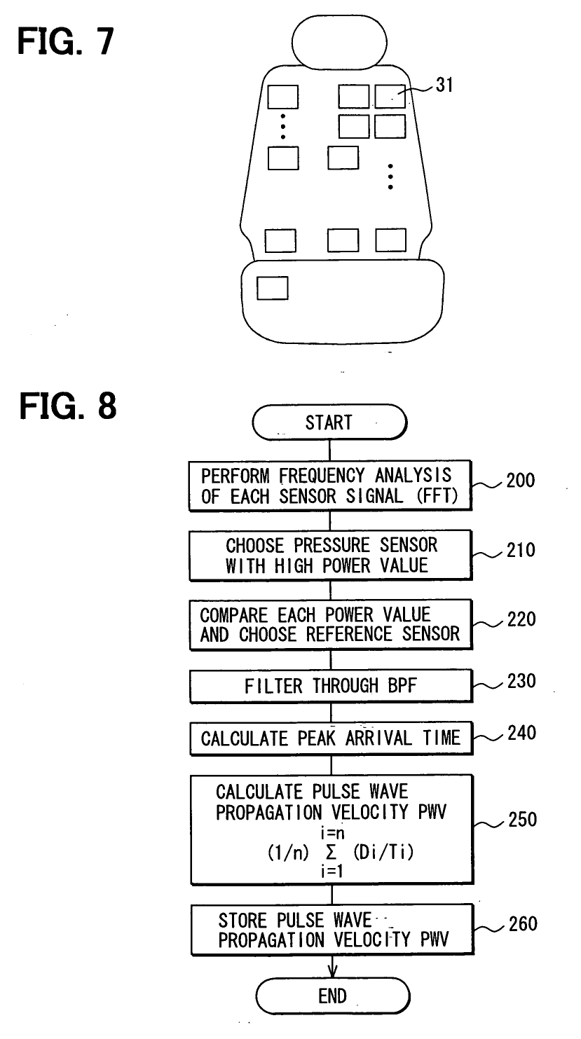

(2) Furthermore, although the detection air bags 7 can be disposed at even intervals in the backrest portion 5 as shown in FIG. 2, detection air bags 31 can be alternatively disposed densely near a part of the backrest portion 5 against which the driver's left shoulder (i.e., the driver's heart) rests as shown in FIG. 7.

This application has the advantage of improving the measurement accuracy since the number of sensor outputs that are added together increases.

(3) While the heart rate waveform is derived from the sensor outputs of the pressure sensors in the first embodiment, a respiratory curve (a respiratory waveform) can be alternatively derived.

In this case, the frequency analysis is performed as shown at step 130 in FIG. 4. Similar to the case of the heart rate, the reference sensor with respect to the breath is chosen from the power spectra indicating condition of the driver's breathing movements that range from 0.15 Hz to 0.4 Hz (at step 140 in FIG. 4). Furthermore, correlation between each pressure sensor is elicited (at step 150 in FIG. 4). According to each sensor output, as shown at step 160 and step 170 in FIG. 4, the sensor outputs are added together (at step 180 that follows) after reversing the phases when the phase differences (τ) are great (e.g., 135° to 225°), whereas the sensor outputs are added together (at step 180) without reversing the phases when the phase differences (τ) are small (e.g., −45° to 45°).

Hence, the respiratory waveform (at step 185 in FIG. 4), and accordingly, the respiratory rate (at step 190 in FIG. 4) are derived from the sensor outputs that have been added up. Ultimately, the driver's drowsiness and/or stress can be assessed at step 195 in FIG. 4.

### Second Embodiment

The second embodiment will be described below, although description, which is similar to that of the first embodiment, is omitted. Since the present embodiment involves a different process from what is described in the first embodiment, content of the process will be described below.

A process of calculating pulse wave propagation velocity PWV that is employed for a process in the present embodiment will be described below. The pulse wave propagation velocity PWV is defined here as average velocity while a pulse wave is propagating and varies between individuals.

In order to detect the pulse wave propagation velocity PWV [m/s] when vehicle vibration is the smallest, for example, when a vehicle idles, the frequency analysis is performed on the sensor output of each pressure sensor by means of FFT at step 200 as shown in a flowchart in FIG. 8.

The power spectra of each sensor signal are derived from results of the frequency analysis. A pressure sensor, a power value of which is high within the range of the heart rate frequency (0.7 to 1.8 Hz), is chosen from those power spectra at step 210. For example, the pressure sensor is chosen if an integral of its power within the range of the heart rate frequency takes the value of a predetermined threshold or higher. Generally, a plurality of such pressure sensors exists.

From a plurality of pressure sensors that have the high power values, the pressure sensor having the highest power value (i.e., the highest integral) is selected as the reference sensor at step 220. Then, at step 230, sensor signals other than the heart rate frequency are attenuated by filtering all sensor outputs of each corresponding pressure sensor that has the high power value through a band-pass filter (BPF; a pass band: 0.7 to 1.8 Hz).

Peak arrival time (Ti) is defined as time that a peak of an output waveform of the reference sensor takes to arrive at a peak of an output waveform of each pressure sensor (except the reference sensor) having the high power value. At step 240, respective peak arrival time (Ti) of all corresponding pressure sensors having the high power values is calculated. That is, the time that indicates a maximum value of a cross-correlation function between the reference sensor and each of these pressure sensors is calculated respectively.

More specifically, each peak arrival time (Ti) can be calculated using the following equations (1), (2) for the cross-correlation function.

$$Rxy ( k ) = ( 1 / N ) {\sum }_{n = 0}^{N - 1 - k} x ( n ) \cdot y i ( n + k ) \qquad ( 1 )$$

x (n): the reference sensor output
y (n): the outputs of the pressure sensors other than the reference sensor
i: identification numbers of the pressure sensors other than the reference sensor
k: the shift amount (time)
N: a maximum value of the shift amount (time)
   Ti=k (the time indicating a maximum value of Rxy (k)) (2)

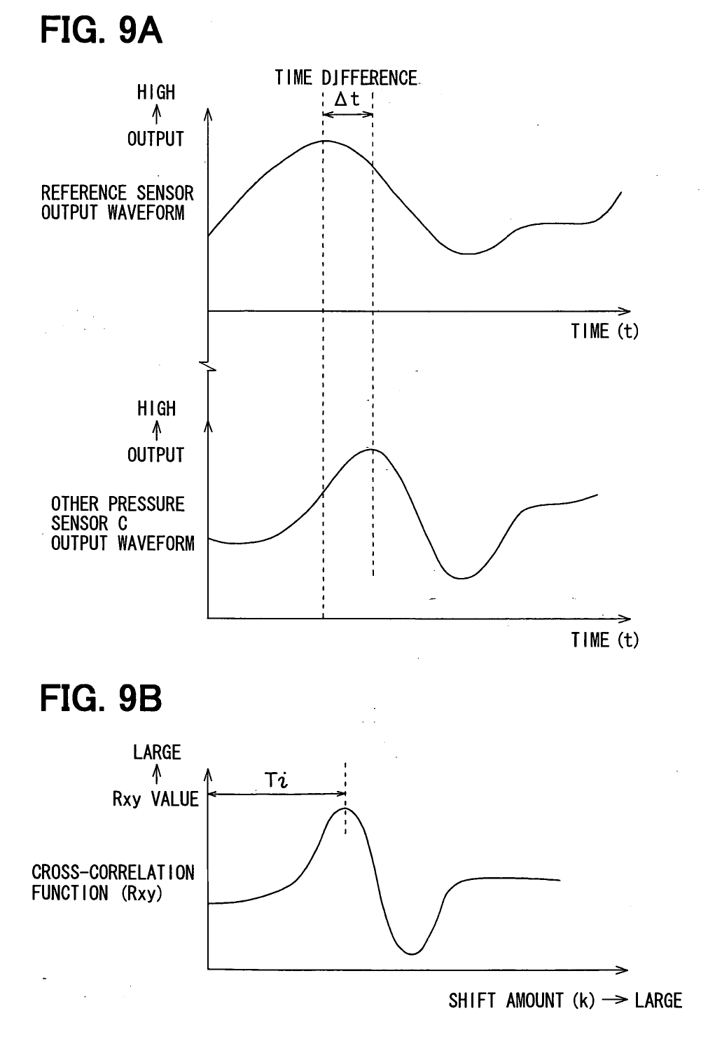

Therefore, as illustrated in FIGS. 9A and 9B, the cross-correlation function Rxy (k) is derived respectively from the reference sensor output x (n) and the outputs yi (n) of the other pressure sensors C by using the above equation (1). Furthermore, k at which the cross-correlation function Rxy (k) is maximized (i.e., the peak arrival time (Ti)) is calculated respectively according to each pressure sensor other than the reference sensor by using the equation (2).

Consequently, the peak arrival time (Ti) of all pressure sensors (except the reference sensor) that have the high power values can be calculated respectively.

At step 250, the pulse wave propagation velocity PWV is derived from all pressure sensors (i=1−n) that have the high power values by using a equation (3) below.

$$PWV = ( 1 / n ) {\sum }_{i = 1}^{i = n} ( T i / D i ) \qquad ( 3 )$$

Di: a distance between the ith pressure sensor and the reference sensor
n the number of pressure sensors that have the high power values
Ti: the pulse wave peak arrival time of the ith pressure sensor from the reference sensor

As regards Di, since a position of each pressure sensor is known, the distance between the reference sensor and the other pressure sensors (that have heart rate elements) can be respectively derived from information about their positions.

At step 260, the pulse wave propagation velocity PWV, which has been calculated using the above equation (3), is stored, and the present process is temporarily completed.

A process of estimating the driver's heart position that is employed in the present embodiment will be described below.

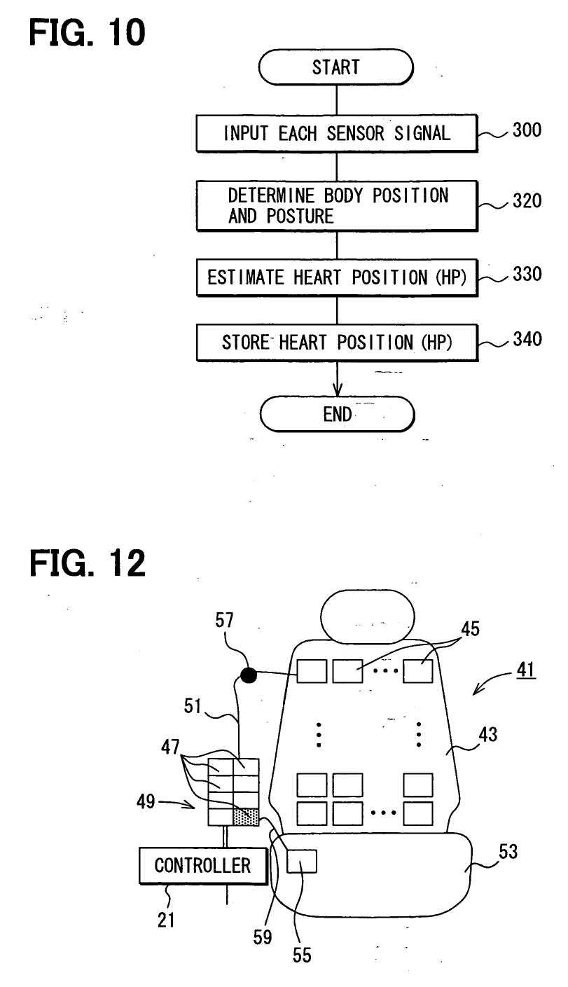

As shown in a flowchart in FIG. 10, data obtained from each pressure sensor is inputted into an input part 21e of the electronic controller 21 (see FIG. 3) at step 300.

At step 320 that follows, the driver's body position and posture are determined based on a signal from each pressure sensor.

More specifically, by means of the cross-correlation function, data obtained as a result of binarization of a pressure value of each pressure sensor is compared with patterns of a body position and posture that have been provided in advance. The measured data that best accords with data on the patterns of the body position and the posture provided beforehand (e.g., when the cross-correlation function is maximized) is defined as the driver's body position and posture at the time.

At step 330, the driver's heart position (HP) is estimated based on the patterns of the body position and the posture determined at step 320. At step 340 that follows, the heart position (HP) estimated at step 330 is stored, and the present process is temporarily completed.

A main process using results of the operation in the present embodiment will be described below.

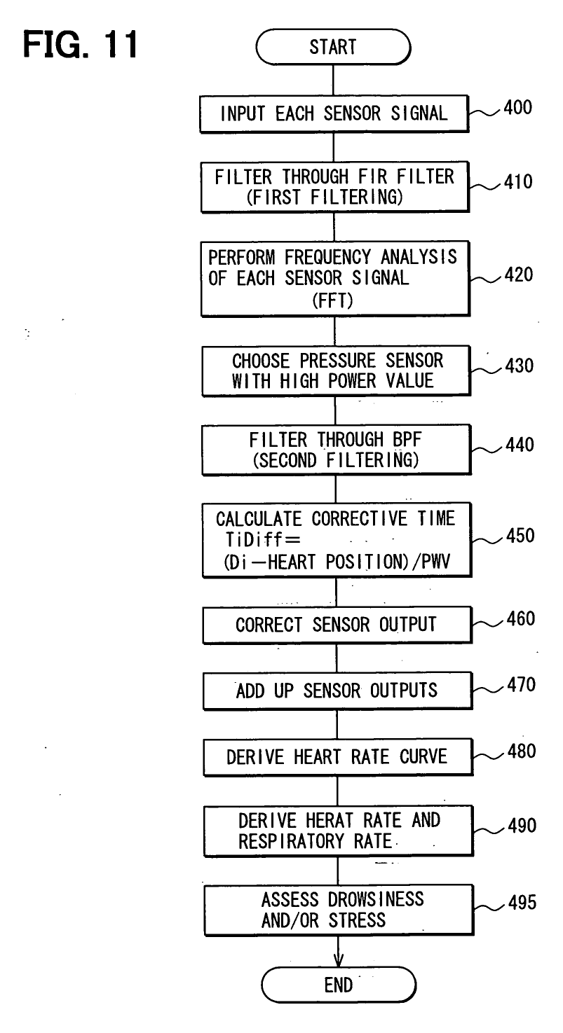

As shown in a flowchart in FIG. 11, data obtained from each pressure sensor is inputted into an input part 21e of the electronic controller 21 (see FIG. 3) at step 400.

At step 410, a first filtering is performed on a sensor signal of each pressure sensor inputted at step 400. More specifically, based on sensor output of the vibration sensor inputted via LAN, the FIR filter is developed by eliciting the adaptive filter coefficient, which is similar to the applications of the first embodiment. The first filtering is performed through this FIR filter.

At step 420, a frequency of a sensor output of each pressure sensor is analyzed by means of FFT. At step 430, the power spectra of each sensor signal are derived from results of the frequency analysis. A pressure sensor a power value of which is higher than a predetermined threshold within the range of the heart rate frequency (0.7 to 1.8 Hz) (e.g., when an integral of the power within the above range is higher than a predetermined threshold) is chosen from those power spectra.

The second filtering is performed at step 440. That is, sensor signals other than the heart rate frequency are attenuated by filtering all sensor outputs of each pressure sensor that has the high power value through the band-pass filter (BPF; the pass band: 0.7 to 1.8 Hz).

At step 450, using an equation (4) below, corrective time TiDiff is derived from the pulse wave propagation velocity PWV and the information about positions of the pressure sensors (Di), which have been obtained through the process in FIG. 8.
TiDiff=(Di−the heart position)/PWV (4)

Therefore, the corrective time TiDiff is calculated by dividing difference between each sensor position (Di) and the heart position obtained through the process in FIG. 10 by the pulse wave propagation velocity PWV.

At step 460, the sensor output of each pressure sensor is corrected by means of the corrective time TiDiff.

More specifically, the correction is carried out by adding the corrective time TiDiff to each sensor output so that each pressure sensor is synchronized with each other.

At step 470, the sensor outputs after the correction are added up.

After this, similar to the first embodiment, the heart rate waveform and the heart rate are derived at steps 480 and 490 respectively from the sensor outputs that have been added up. Lastly, the driver's drowsiness and/or stress are assessed at step 495.

Through the process as described above, the present embodiment has a similar effect to the effect described in the first embodiment. Furthermore, the sensor outputs are corrected according to the heart position, which has been estimated based on the patterns of the driver's body position and posture. Consequently, differences between the sensor outputs can be appropriately corrected, which leads to improved accuracy of measurement of, for example, the heart rate or the like.

Besides, the pulse wave propagation velocity PWV is used for calculation of the corrective time TiDiff to correct the sensor outputs. Hence, there is an advantage of reducing influence of differences of the pulse wave propagation velocity PWV between individuals.

### Third Embodiment

The third embodiment will be described below, although description, which is similar to that of the first embodiment, is omitted.

Similar to the first embodiment, in a backrest portion 43 of a driver's seat 41, a plurality of detection air bags 45 are arrayed in a grid pattern as shown in FIG. 12.

A sensor array 49 aside of the driver's seat 41 includes sensor elements 47 that are arrayed in a grid pattern. Via an air tube 51, each detection air bag 45 is connected to each sensor element 47 in such a manner that the detection air bag 45 and the sensor element 47 correspond one-to-one to each other. In addition, reference air bags 55 similar to those in the first embodiment are arrayed in a seating face portion 53

In the present embodiment particularly, a pressure valve 57 is inserted in an air tube 51 that connects each detection air bag 45 to the corresponding sensor element 47.

By opening under certain conditions, for example, when pressure applied to the valve 57 becomes equal to or greater than a predetermined level within a unit time, the pressure valve 57 reduces the pressure inside the air tube 51 (and thus, the pressure applied to the sensor element 47).

By virtue of the pressure valve 57, a signal that is far stronger than a biological signal (that indicates the driver's heart rate in this case) can be excluded, thereby improving the measurement accuracy.

The present invention is not by any means limited to the above embodiments, and it is apparent that it can be embodied in many ways without departing from the scope of the invention.

(1) Accuracy of the sensor outputs of the pressure sensors placed near the heart position may be higher than that of the pressure sensors placed not close to the heart position. Hence, after the driver's heart position is estimated, the sensor outputs of the pressure sensors that are placed within a defined distance from the heart position can only be employed, for example. Alternatively, weighting can be performed on the sensor outputs. That is, the sensor outputs of the pressure sensors within the defined distance can be increased, or conversely, the sensor outputs beyond the defined distance can be decreased. By means of these operations, the measurement accuracy will be improved.

(2) For example, as a result of the frequency analysis of the sensor outputs, the sensor outputs of the pressure sensors that detect biological frequency elements such as the heart rate frequency or the like can only be employed. As a result, influence of noise can be minimized, and the measurement accuracy will be improved.

Additional advantages and modifications will readily occur to those skilled in the art. The invention in its broader terms is therefore not limited to the specific details, representative apparatus, and illustrative examples shown and described.

What is claimed is:

1. A biosignal detection device comprising:
a plurality of pressure sensors that are arranged at a seat of a vehicle to sense a pressure of a body of a vehicle occupant when the vehicle occupant is present on the seat; and
a controller that detects biosignal, which is relevant to a human body activity of the vehicle occupant present on the seat, based on a measurement of at least one effective pressure sensor, which is selected from the plurality of pressure sensors in such a manner that the measurement of the at least one effective pressure sensor is less than a first predetermined pressure and is greater than a second predetermined pressure, wherein the second predetermined pressure is less than the first predetermined pressure.

2. The biosignal detection device according to claim 1, wherein the plurality of pressure sensors is densely arranged at a portion of the seat, which corresponds to a position of a heart of the vehicle occupant when the vehicle occupant is present on the seat.

3. The biosignal detection device according to claim 1, wherein:
the at least one effective pressure sensor includes multiple effective pressure sensors; and
the controller estimates a position of a heart of the vehicle occupant based on the measurements of the multiple effective pressure sensors.

4. The biosignal detection device according to claim 3, wherein the controller estimates the position of the heart of the vehicle occupant by comparing the measurements of the multiple effective pressure sensors with corresponding data, which indicates a previously obtained body position and posture pattern.

5. The biosignal detection device according to claim 3, wherein the controller corrects the measurement of at least one of the multiple effective pressure sensors based on the estimated position of the heart.

6. The biosignal detection device according to claim 3, wherein the controller selects the multiple effective pressure sensors from the plurality of pressure sensors based on information that indicates the position of the heart of the vehicle occupant.

7. The biosignal detection device according to claim 1, wherein the controller selects the at least one effective pressure sensor from the plurality of pressure sensors in such a manner that the measurement of the at least one effective pressure sensor indicates a human body frequency component, which indicates the human body activity.

8. The biosignal detection device according to claim 1, wherein the controller senses a vibration caused by a factor that is not relevant to the human body activity based on at least one vibration reference pressure sensor, which is selected from the plurality of pressure sensors and is arranged at a seating face portion of the seat and is other than the at least one effective pressure sensor.

9. The biosignal detection device according to claim 8, wherein:
the controller includes an adaptive filter, which cancels the vibration caused by the factor that is not relevant to the human body activity; and
the controller sets at least one parameter of the adaptive filter based on a measurement of the at least one vibration reference pressure sensor.

10. The biosignal detection device according to claim 9, wherein the controller filters the measurement of the at least one effective pressure sensor through the adaptive filter.

11. The biosignal detection device according to claim 1, further comprising at least one vibration sensor, each of which is arranged in the vehicle and is used as a vibration reference pressure sensor that senses a vibration caused by a factor that is not relevant to the human body activity.

12. The biosignal detection device according to claim 11, wherein:
the controller includes an adaptive filter, which cancels the vibration caused by the factor that is not relevant to the human body activity; and
the controller sets at least one parameter of the adaptive filter based on a measurement of the at least one vibration reference pressure sensor.

13. The biosignal detection device according to claim 12, wherein the controller filters the measurements of the multiple effective pressure sensors through the adaptive filter.

14. The biosignal detection device according to claim 1, wherein:
the at least one effective pressure sensor includes multiple effective pressure sensors; and
the controller corrects the measurement of at least one of the multiple effective pressure sensors based on a phase difference between the measurement of the at least one of the multiple effective pressure sensors and another one of the multiple effective pressure sensors.

15. A biosignal detection device comprising:
a plurality of pressure sensors that are arranged at a seat of a vehicle to sense a pressure of a body of a vehicle occupant when the vehicle occupant is present on the seat; and
a controller that detects biosignal, which is relevant to a human body activity of the vehicle occupant present on the seat, based on measurements of multiple effective pressure sensors, which are selected from the plurality of pressure sensors, wherein the controller estimates a position of a heart of the vehicle occupant based on the measurements of the multiple effective pressure sensors when the vehicle occupant is present on the seat.

16. The biosignal detection device according to claim 15, wherein the controller estimates the position of the heart of the vehicle occupant by comparing the measurements of the multiple effective pressure sensors with corresponding data, which indicates a previously obtained body position and posture pattern.

17. The biosignal detection device according to claim 15, wherein the controller corrects the measurement of at least one of the multiple effective pressure sensors based on the estimated position of the heart.

18. The biosignal detection device according to claim 15, wherein the controller selects the multiple effective pressure sensors from the plurality of pressure sensors based on information that indicates the position of the heart of the vehicle occupant.

19. The biosignal detection device according to claim 15, wherein the controller selects the multiple effective pressure sensors from the plurality of pressure sensors in such a manner that the measurements of the multiple effective pressure sensors indicate a human body frequency component, which indicates the human body activity.

20. The biosignal detection device according to claim 15, wherein the controller senses a vibration caused by a factor that is not relevant to the human body activity based on at least one vibration reference pressure sensor, which is selected from the plurality of pressure sensors and is arranged at a seating face portion of the seat and is other than the multiple effective pressure sensors.

21. The biosignal detection device according to claim 20, wherein:
the controller includes an adaptive filter, which cancels the vibration caused by the factor that is not relevant to the human body activity; and
the controller sets at least one parameter of the adaptive filter based on a measurement of the at least one vibration reference pressure sensor.

22. The biosignal detection device according to claim 20, wherein the controller filters the measurements of the multiple effective pressure sensors through the adaptive filter.

23. The biosignal detection device according to claim 15, further comprising at least one vibration sensor, each of which is arranged in the vehicle and is used as a vibration reference pressure sensor that senses a vibration caused by a factor that is not relevant to the human body activity.

24. The biosignal detection device according to claim 23, wherein:
the controller includes an adaptive filter, which cancels the vibration caused by the factor that is not relevant to the human body activity; and
the controller sets at least one parameter of the adaptive filter based on a measurement of the at least one vibration reference pressure sensor.

25. The biosignal detection device according to claim 24, wherein the controller filters the measurements of the multiple effective pressure sensors through the adaptive filter.

26. The biosignal detection device according to claim 15, wherein the controller corrects the measurement of at least one of the multiple effective pressure sensors based on a phase difference between the measurement of the at least one of the multiple effective pressure sensors and another one of the multiple effective pressure sensors.
